(19) United States
(12) Patent Application Publication
(10) Pub. No.: US 2025/0010078 A1
(43) Pub. Date: Jan. 9, 2025

(54) SYSTEMATIC OPTIMIZATION OF STIMULATION SETTINGS FOR DEEP BRAIN STIMULATION TREATMENT OF EPILEPSY

(71) Applicant: Regents of the University of Minnesota, Minneapolis, MN (US)

(72) Inventors: Theoden I. Netoff, Minneapolis, MN (US); Robert McGovern, III, Minneapolis, MN (US); Zachary Sanger, Minneapolis, MN (US); Hafsa Farooqi, Minneapolis, MN (US); David Darrow, Minneapolis, MN (US)

(21) Appl. No.: 18/763,558

(22) Filed: Jul. 3, 2024

Related U.S. Application Data
(60) Provisional application No. 63/524,773, filed on Jul. 3, 2023.

(51) Int. Cl. A61N 1/36; A61N 1/05; A61N 1/372; G16H 20/40

(52) U.S. Cl. CPC A61N 1/36139; A61N 1/0534; A61N 1/36064; A61N 1/3615; A61N 1/36171; A61N 1/36175; A61N 1/37247; G16H 20/40

(57) ABSTRACT

Described here are systems and methods for testing neurostimulation settings and measuring their effects on neural activity, which may then be used to select subject-specific neurostimulation settings. In general, the present disclosure provides systems and methods that utilize neural recordings to help determine the neurostimulation settings that maximally reduce a particular neural activity.

### CROSS-REFERENCE TO RELATED APPLICATIONS

This application claims the benefit of U.S. Provisional Patent Application Ser. No. 63/524,773, filed on Jul. 3, 2023, and entitled “SYSTEMATIC OPTIMIZATION OF STIMULATION SETTINGS FOR DEEP BRAIN STIMULATION TREATMENT OF EPILEPSY,” which is herein incorporated by reference in its entirety.

### STATEMENT OF FEDERALLY SPONSORED RESEARCH

This invention was made with government support under NS124616 awarded by the National Institutes of Health. The government has certain rights in the invention.

### BACKGROUND

Neurostimulation modulates the neural activity of a subject's nervous system, such as by applying electrical and/or magnetic fields to one or more anatomical targets in the subject's nervous system. The application of neurostimulation can be used to provide motor cortex stimulation, cranial nerve stimulation (e.g., vagus nerve stimulation), spinal cord stimulation, as well as treatment for neurological disorders, psychiatric disorders, and the like.

Deep brain stimulation (DBS) is used to treat epilepsy. The implanted stimulators have parameters, such as stimulation frequency, pulse width, and amplitude, which can be changed remotely using a clinical programmer. Currently, one stimulation frequency and pulse width are used clinically, but there is little evidence that this setting is optimal, or that if the setting is optimal for some patients, it is optimal for all patients. There is no process for tuning stimulation settings to improve patient outcome. New stimulators, allow recording of neural activity on electrodes that are not used for stimulation.

### SUMMARY OF THE DISCLOSURE

It is an aspect of the present disclosure to provide a method for determining neurostimulation settings for a neurostimulation device. The method includes measuring neural activity in a subject using a recording electrode while delivering neurostimulation using a stimulating electrode. The neurostimulation is delivered according to a plurality of different neurostimulation settings, where each of the plurality of different neurostimulation settings may include a different set of stimulation parameters. Each different set of stimulation parameters may include different values for a stimulation frequency, a pulse width, and an amplitude. A subject-specific neurostimulation setting is determined using a computing device based on the measured neural activity by: estimating a power for each neurostimulation setting from the neural activity measured while neurostimulation was being delivered with that neurostimulation setting; generating a response surface from the power estimated for each neurostimulation settings, where the response surface indicates estimated power as a function of the neurostimulation settings; and selecting the subject-specific neurostimulation setting based on the response surface. The subject-specific neurostimulation setting is then outputted from the computing device to the neurostimulation device. Other embodiments of this aspect include corresponding systems (e.g., computer systems), programs, algorithms, and/or modules, each configured to perform the steps of the methods.

It is another aspect of the present disclosure to provide a non-transitory computer-readable storage medium having stored thereon instructions that when executed by a processor cause the processor to: (i) control a neurostimulation device to deliver neurostimulation to a subject using a stimulating electrode, where the neurostimulation is delivered according to neurostimulation settings may include a stimulation frequency, a pulse width, and an amplitude; (ii) control the neurostimulation device to measure neural activity data from the subject using a recording electrode, where the neural activity data may include local field potentials measured in response to the delivered neurostimulation; (iii) repeat steps (i) and (ii) while adjusting the neurostimulation settings by adjusting at least one of the stimulation frequency, the pulse width, or the amplitude; (iv) generate a response surface from the neural activity data, where the response surface indicates measured neural activity as a function of the neurostimulation settings; (v) determine an updated neurostimulation setting based on a Bayesian optimization using the response surface; and (vi) store the updated stimulation setting in a memory of the neurostimulation device. Other embodiments of this aspect include corresponding systems (e.g., computer systems), programs, algorithms, and/or modules, each configured to perform the steps of the methods.

### BRIEF DESCRIPTION OF THE DRAWINGS

FIG. 1 illustrates an example workflow for determining optimal neurostimulation settings based on neural activity measured while delivering neurostimulation.

FIG. 2 illustrates example LFP time traces and spectrograms.

FIG. 3 illustrates another example of LFP time traces and spectrograms.

FIG. 4 illustrates power measured in a 1-80 Hz band in the right hemisphere of a subject over a grid of three different frequencies and three different pulse widths.

FIG. 5 illustrates measured errors over a number of different test batches.

FIG. 6 illustrates an example 10-minute average and 2-hour smoothed data for 130 days from an epilepsy patient. Inset shows cycle of power that occurs on daily cycle. Top right shows average power by hour of day. High amplitude events (>80 dB) are also plotted as a function of day. Events on left side of the brain show an increase over hours 15-24, during patient's typical seizure occurrences.

FIG. 7 shows an example graphical user interface (GUI) that can be used to load in measured neural activity data and implementing the Bayesian optimization for suggesting new neurostimulation settings.

FIG. 8 illustrates a flowchart of an example method for determining subject-specific neurostimulation settings based on a Bayesian optimization of neural activity measured in response to a plurality of different neurostimulation settings.

FIG. 9 is a block diagram of an example controller that can implement the systems and methods described in the present disclosure.

FIG. 10 illustrates an example neurostimulation device.

FIG. 11 is a block diagram of an example system for generating, determining, or otherwise selecting subject-specific neurostimulation settings in accordance with some embodiments described in the present disclosure.

FIG. 12 is a block diagram of example components that can implement the system of FIG. 11.

### DETAILED DESCRIPTION

Described here are systems and methods for testing stimulation settings in the clinic, and measuring their effects on neural activity, which may be used to select stimulation settings. In general, the present disclosure provides systems and methods that utilize neural recordings to help determine the stimulation settings that maximally reduce a particular neural activity. The disclosed algorithm can be used as a recommender system separate from any particular neurostimulation device.

In general, the systems and methods select settings that use neural recordings and Bayesian optimization to suggest new settings for testing. This process can help guide a physician, technician, or other user through the process of testing different settings to find the setting that maximally benefits the patient to suppress neural activity measured in the brain during stimulation, and eventually to alleviate their seizures or other neurological condition.

Deep brain stimulation (DBS) is used to treat other diseases, such as Parkinson's disease and essential tremor. While the examples described in the present disclosure are related to optimizing stimulation settings for epilepsy, the techniques can be readily adapted to apply them to Parkinson's, essential tremor, and other applications for which DBS can be used as a therapy, such as chronic pain, depression, and obsessive compulsive disorder.

As a non-limiting example, DBS settings are optimized using neural feedback for the treatment of epilepsy of other neurological conditions. For instance, neural activity in the thalamus can change with stimulation parameters, which may be used to optimize settings.

Electrical stimulation of the superior anterior nucleus of the thalamus (SANTE) has been shown to be clinically effective for suppressing seizures. Stimulation frequency of 145 Hz and 90 μsec at 1 mA can be used as a default setting, but it is not clear if this is the optimal setting for every patient. In some configurations, a neurostimulation device can be configured to allow for simultaneous stimulation and recording, allowing for the observation of how the activity in the thalamus, or other brain region, depends on stimulation settings.

In an example study, in epileptic patients implanted with such a neurostimulation device, thalamic activity was measured while systematically stimulating over a grid of three frequencies and three pulse widths while adjusting the stimulation amplitude, so that energy was kept constant (e.g., for consistent energy delivery across parameter combinations). It has been found that activity in the thalamus changes significantly with different stimulation parameters and the findings are reproducible across visits. Furthermore, the setting that minimizes thalamic activity is often not the setting being used clinically. It is contemplated that this systematic review of thalamic activity across stimulation settings in clinic may be used to identify settings that could further improve patient outcomes.

The neurostimulation device used in this example study included a neurostimulator that is equipped with sensing, allowing recording from electrodes neighboring stimulation electrodes to record neural activity at 250 Hz, or the like. In use, stimulating electrodes were placed in the superior anterior nucleus of the thalamus for treatment of epilepsy. Default stimulation was at 145 Hz, with 90 μsec pulses at 1 mA for one minute on and four minutes off.

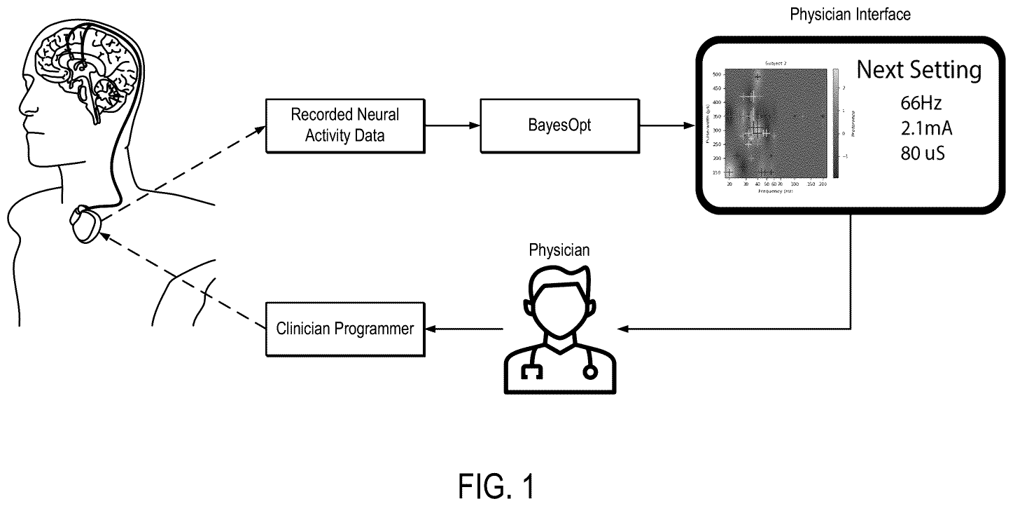

Patients implanted with the neurostimulation device were stimulated with DBS applied to the superior anterior nucleus of the thalamus at a selected stimulation frequency, pulse width, and amplitude. The local field potential measured from the thalamus is transmitted during stimulation to a Bayesian optimization (BayesOpt) system, as shown in FIG. 1. Stimulus artifacts can be removed and the power at that setting can be estimated from a one-minute segment of recorded data. BayesOpt will then display a response surface showing the measured power as a function of the stimulation parameters and suggest a new setting for programming. The clinician, or other user, can program the patient's stimulator with the new setting. This testing loop can be repeated for 20 settings to optimize stimulation settings.

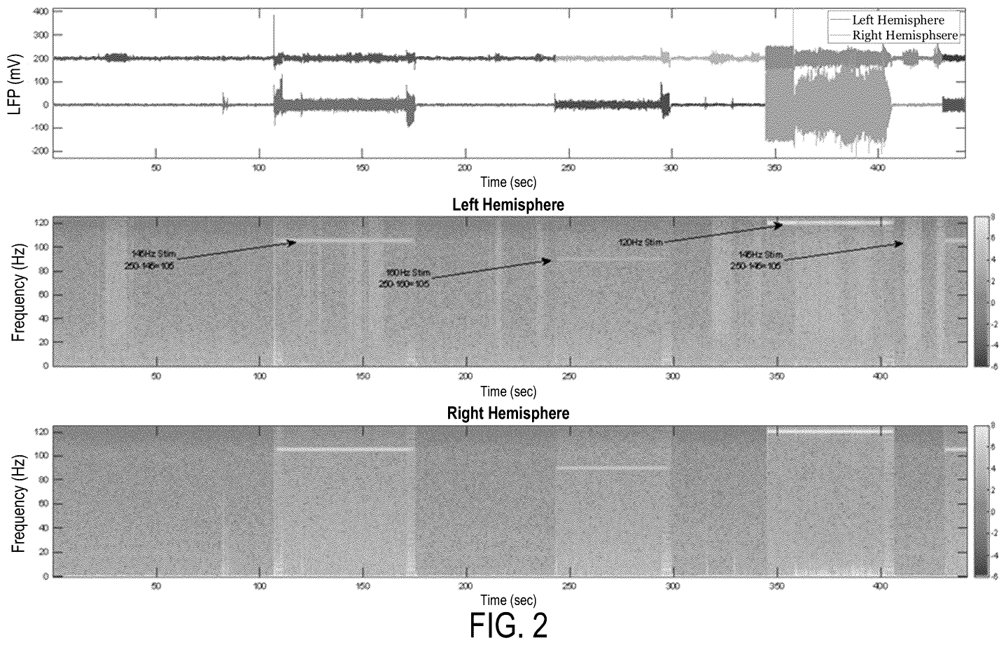

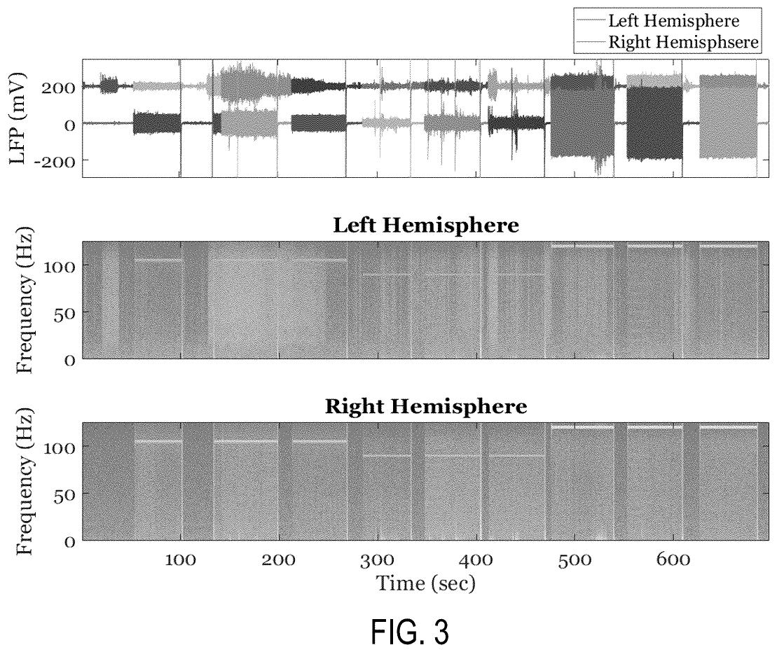

As shown in FIGS. 2 and 3, neural activity recordings show stimulus artifacts in time trace (top) and spectrogram (middle, bottom) limited to a very narrow band and neural activity is clearly isolatable. Stimulus artifacts can be seen in time and spectrogram, but the artifacts are frequency band limited. 145 Hz and 160 Hz stim frequencies are above the Nyquist frequency of 250 Hz sampling and are therefore aliased. Power measured in the α-band, β/γ-band, and broadband show strong differences in measured power at different stimulation frequencies. The power measured during stimulation in clinic can be used to guide programming by using stimulation settings that maximally suppress activity.

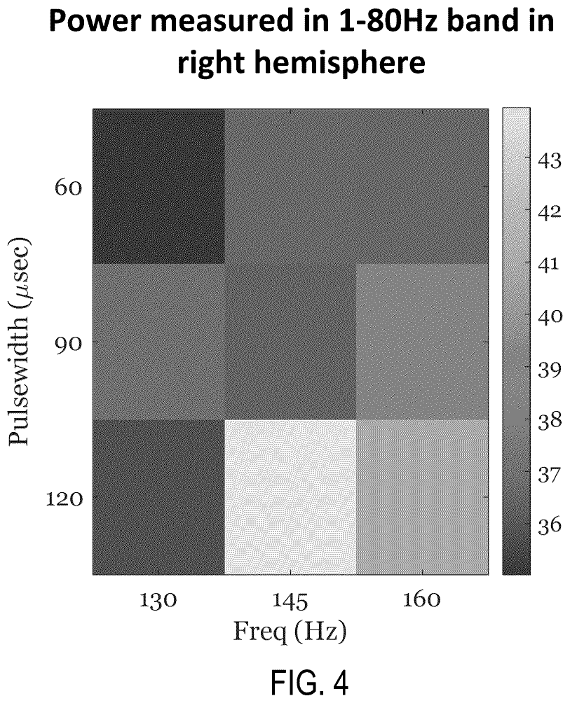

In the illustrated example, nine different stimulation settings were applied: three frequencies by three pulse-widths, centered around the standard clinical setting of 145 Hz and 90 μseconds. Amplitude was adjusted so that total energy delivered was the same under all stimulation settings. Power measured between 1 and 80 Hz was measured and total power is shown in FIG. 4. It can be seen that 120 Hz stimulation at 60 Hz had the lowest power measured in the right hemisphere.

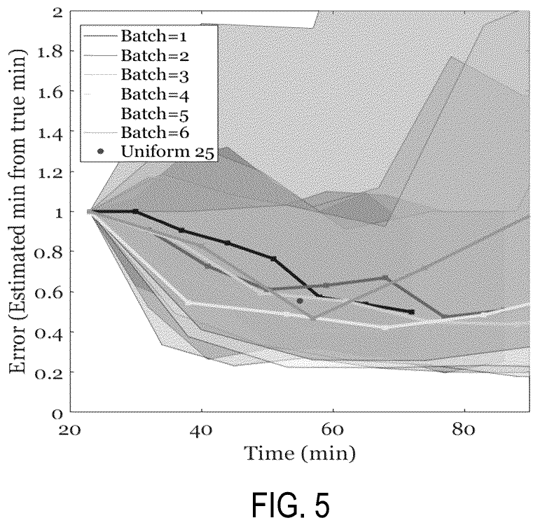

The neural activity data are analyzed and the results used to generate a response surface. Bayesian optimization can then be used to select the next optimal settings for testing. After data are downloaded from the clinical programmer for analysis, the settings can be tested in batches (e.g., in a 1-hour clinical visit). As an example, optimization can be tested on a surface with different batch sizes. In an example study, batches of three produced good results in a 1-hour visit. An example of estimated errors in various batches is illustrated in FIG. 5.

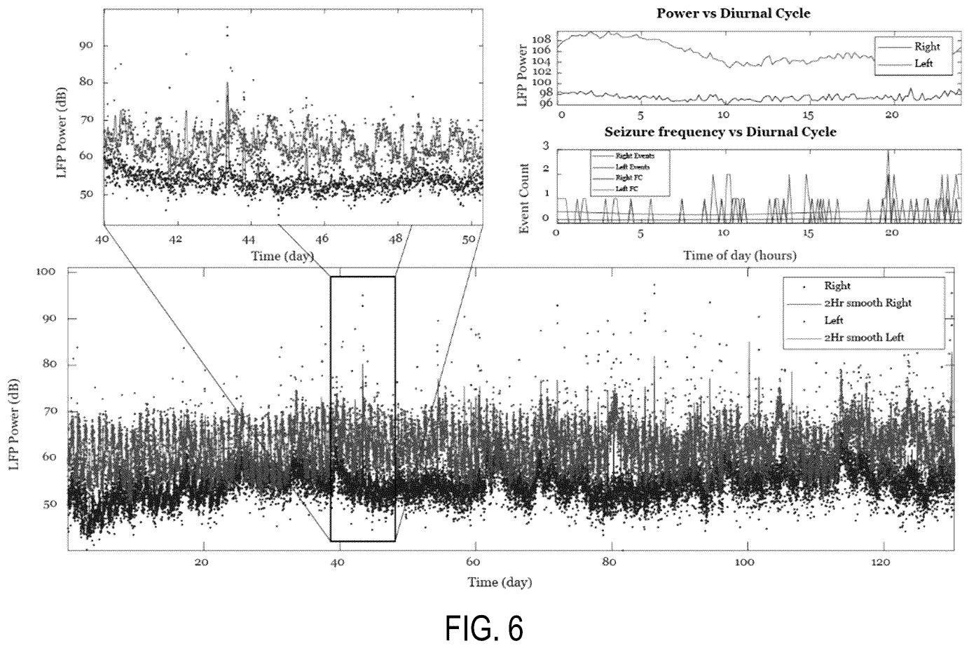

In some configurations, the neurostimulation device records 10-minute averages of power in selected frequency bands and stores the data for download, as illustrated in FIG. 6. The bottom of FIG. 6 shows 10-minute average and 2-hour smoothed data for 130 days from an epilepsy patient. The inset shows cycle of power that occurs on daily cycle. The top right of FIG. 6 shows average power by hour of day. High amplitude events (>80 dB) are also plotted as a function of day. Events on left side of the brain show an increase over hours 15-24, during patient's typical seizure occurrences.

Neural recordings with a neurostimulation device are advantageous and neural activity can be measured while stimulation is applied. Neural activity changes significantly with stimulation frequency and pulse width, while compensating with amplitude to keep total energy the same. Bayesian optimization with batches of three settings can be used to efficiently find settings that minimize activity within a 1-hour visit. Recorded averages of the LFP power in the alpha band every 10 minutes can be used to assess the effect of the optimized settings over the standard clinical setting. Preliminary LFP recordings show strong diurnal cycles of activity and events that peak at times when patient reports highest likelihood of seizures.

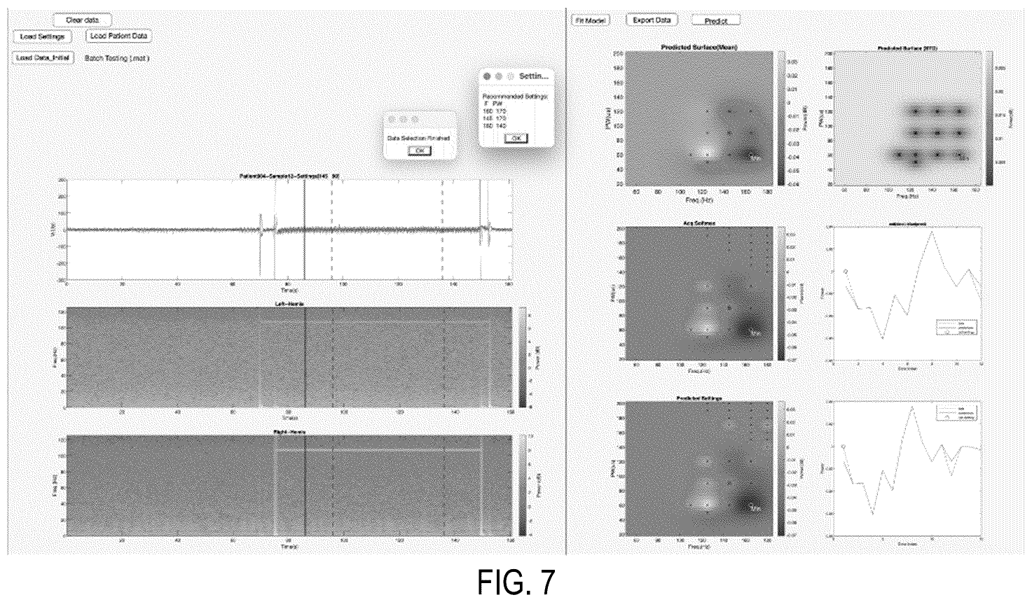

FIG. 7 shows an example graphical user interface (GUI) that can be used to load in measured neural activity data and implementing the Bayesian optimization for suggesting new neurostimulation setting.

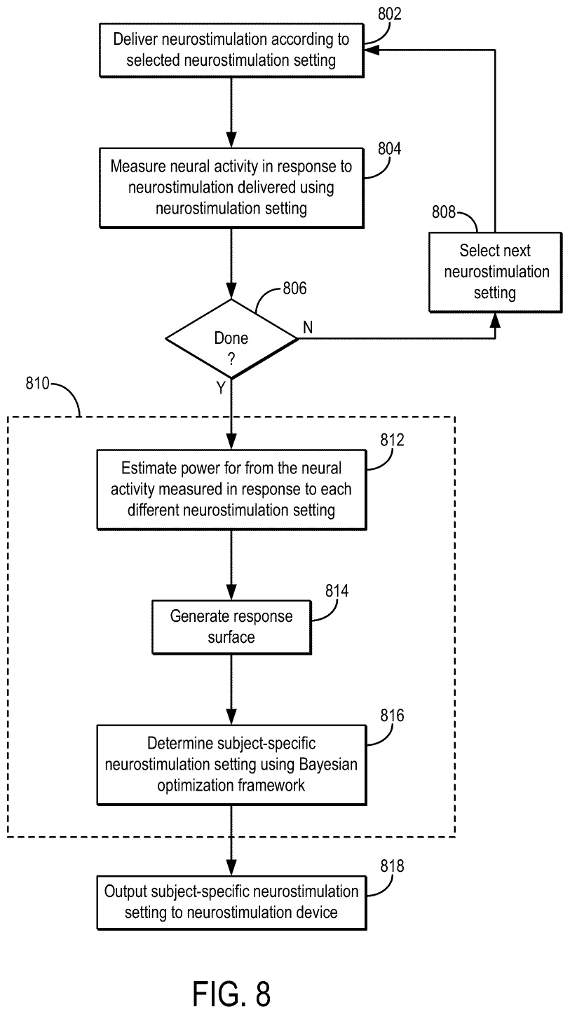

FIG. 8 illustrates a flowchart setting forth the steps of an example method for determining, or otherwise selecting, a subject-specific neurostimulation setting to control the delivery of neurostimulation to the subject using a neurostimulation or other neuromodulation device.

The method includes delivering neurostimulation to a subject using one or more stimulation electrodes of a neurostimulation device, as indicated at step 802. Alternatively, other neuromodulation can be delivered to the subject using a suitable neuromodulation device. The neurostimulation is delivered to the subject according to a plurality of different neurostimulation settings. Each neurostimulation setting includes a different set of stimulation parameters, such that the different neurostimulation settings correspond to delivering a different neurostimulation to the subject. The sets of stimulation parameters each include different values for a stimulation frequency, a pulse width, and/or an amplitude.

The different neurostimulation settings therefore include different values of stimulation parameters including stimulation frequency, pulse width, and/or amplitude. In some implementations, the different neurostimulation settings can be selected to search a grid of potential stimulation parameters. For instance, the neurostimulation settings can include systematic adjustments to stimulation frequency and/or pulse width. The amplitude in each of the different sets of stimulation parameters can be selected to maintain a constant energy delivery across the plurality of different neurostimulation settings. For example, the amplitude for a neurostimulation setting can be selected based on the different values for the stimulation frequency and the pulse width in that neurostimulation setting, such that the resulting energy delivery associated with that neurostimulation setting is constant, or relatively constant, with respect to the other neurostimulation settings used to deliver neurostimulation to the subject.

While the neurostimulation is being delivered to the subject according to the different neurostimulation settings, neural activity is recorded from the subject using one or more recording electrodes, as indicated at step 804. The measured neural activity can include recordings of electrical activity in the subject's brain measured in response to the different neurostimulation settings used to deliver neurostimulation to the subject. In this way, the measured neural activity is indicative of changes in neural activity in response to the neurostimulation being delivered to the subject with the different neurostimulation settings. The neural activity data may include, for example, measurements of local field potentials (LFPs) recorded using the one or more recording electrodes.

The neural activity can be recorded from one or more locations in the subject's brain. For instance, the neural activity may be recorded from a particular region of the subject's brain. As described above, one such brain region is the thalamus. In these instances, the neural activity may be recorded from the subject's thalamus, or a portion thereof (e.g., the superior anterior nucleus of the thalamus). The measured neural activity indicates the thalamic activity of the subject in response to the neurostimulation being delivered with the different neurostimulation settings. In other implementations, the neural activity can be recorded from brain regions other than the thalamus.

As indicated at decision block 806, after neural activity is measured in response to neurostimulation delivered using a selected neurostimulation setting, a determination is made whether additional neurostimulation settings should be evaluated. If so, then the next neurostimulation setting is selected at step 808 and steps 802 and 804 are repeated with the different neurostimulation setting to measure additional neural activity in response to the different neurostimulation settings.

Once all of the neurostimulation settings have been tested, the process proceeds at decision block 806 to determine a subject-specific neurostimulation setting, as indicated at process block 810. As described above, the subject-specific neurostimulation setting can be selected using a Bayesian optimization framework to analyze the measured neural activity.

In some embodiments, determining or otherwise selecting the subject-specific neurostimulation setting can include the following substeps. First, a power is estimated for each neurostimulation setting from the neural activity measured while neurostimulation was being delivered with that neurostimulation setting, as indicated at step 812. Using the power estimated for each neurostimulation setting, a response surface can be generated, as indicated at step 814. The response surface indicates estimated power as a function of the neurostimulation settings. As a non-limiting example, the response surface may be modeled using a Gaussian process, or the like.

A Bayesian optimization framework can then be implemented using the response surface to select a subject-specific neurostimulation settings, as indicated at step 816. The response surface is used to guide a Bayesian optimization process by using the response surface as a surrogate model of an objective function to be optimized. The Bayesian optimization framework can use the response surface to identify promising regions in the input space, which helps select the optimal neurostimulation setting for the subject.

The subject-specific neurostimulation setting can be selected to minimize neural activity in a particular region of the subject's brain. For instance, the subject-specific neurostimulation setting can be selected to minimize neural activity in the subject's thalamus, or a portion thereof (e.g., the superior anterior nucleus of the thalamus).

The subject-specific neurostimulation setting is then output to the neurostimulation device, as indicated at step 818. For instance, the subject-specific neurostimulation setting is sent to the neurostimulation device where it can be stored in a memory of the neurostimulation device. The neurostimulation device can then retrieve the subject-specific neurostimulation setting from its memory and use the subject-specific neurostimulation setting to deliver neurostimulation to the subject, which has been optimized to achieve a desired therapeutic effect in the subject.

In an example implementation, the process of determining or otherwise selecting the subject-specific neurostimulation setting can therefore include defining an objective function to be optimized through the Bayesian optimization framework. The objective function may generally model how changes in stimulation parameters in the neurostimulation setting will affect the measured neural activity. A prior distribution can then be generated for the objective function. The prior can be generated using a Gaussian process, or the like. The prior can be initialized with a suitable kernel. As a non-limiting example, the prior can be a Gaussian process that is initialized with a radial basis function kernel. The measured neural activity and the corresponding neurostimulation settings used to deliver neurostimulation while the neural activity was being measured are then mapped to the prior distribution to obtain a posterior distribution. An acquisition function can then be used to select the next neurostimulation setting to test. The objective function can then be evaluated using this updated neurostimulation setting and the estimate of the responsive neural activity can then be used to update the Gaussian process model. This process of evaluating new neurostimulation settings can be iterated until a stopping criterion is satisfied (e.g., the estimated neural activity is minimized).

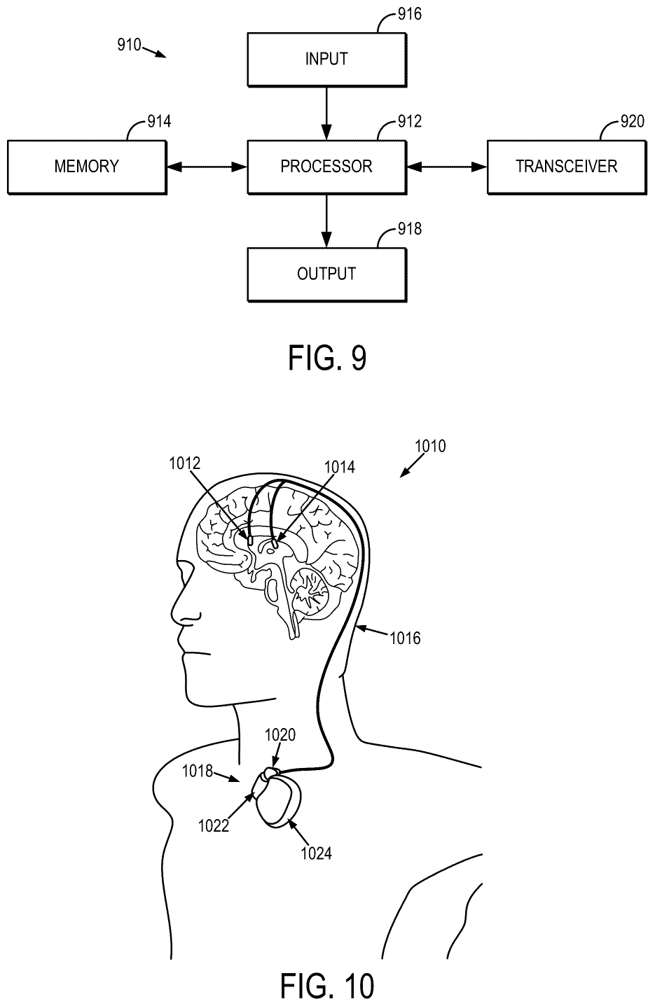

Referring now to FIG. 9, an example of a controller 910 that can implement the methods described in the present disclosure to control a neurostimulation device is illustrated. In general, the controller 910 includes a processor 912, a memory 914, and input 916, and an output 918. The controller 910 can be implemented as part of a neurostimulation device, or as a separate controller that is in communication with the neurostimulation device via the output 918. As one example, the controller 910 can be implemented in a neurostimulation device, such as an implantable medical device (e.g., an implanted neurostimulation system such as a deep brain stimulation (DBS) system), a standalone neurostimulation device (e.g., an external neurostimulation device, such as a transcranial magnetic stimulation (TMS) system, transcranial electrical stimulation (tES) system), and so on. In other examples, the controller 910 can be implemented in a remote computer that communicates with the neurostimulation device. In still other examples, the controller 910 can be implemented in a smartphone that is paired with the neurostimulation device, such as via Bluetooth or another wireless or wired communication.

In some embodiments, the input 916 is capable of recording neural signal data, or other physiological measurement data, from the user. As one example, the neural signal data can be electrophysiological activity data (e.g., EEG signal data, other measured neural activity data), and the input 916 can be one or more electrodes (e.g., external electrodes, implanted electrodes). The input 916 can include a wired or wireless connector for receiving neural signal data. These neural signal data can be transmitted to the controller 910 via the input 916.

The processor 912 includes at least one hardware processor to execute instructions embedded in or otherwise stored on the memory 914 to implement the methods described in the present disclosure. The memory can also store baseline signal data, measured neural signal data, one or more models, and model parameters, as well as settings to be provided to the processor 912 for generating control signals to be provided to a neurostimulation device via the output 918.

The output 918 communicates control signals to a neurostimulation device. As one example, the control signals provided to the output 918 can control one or more electrodes and/or electromagnetic coils to operate under control of the controller 910 to deliver electromagnetic stimulations (e.g., by generating electric fields, magnetic fields, or both) to the subject. Circuitry in the controller 910 can detect and processes electrophysiological activity sensed by the one or more electrodes via the input 916 to determine and/or adjust stimulation settings (e.g., stimulation waveforms) based on the methods and algorithms described in the present disclosure. The stimulation settings are provided as instructions to a pulse generator in the neurostimulation device via the output 918, which in response to the instructions provides an electrical signal to the one or more electrodes or electromagnetic coils to deliver the neurostimulations to the subject.

The controller 910 can also include a transceiver 920 and associated circuitry for communicating with a programmer or other external or internal device. As one example, the transceiver 920 can include a telemetry coil. In some embodiments, the transceiver 920 can be a part of the input 916.

In operation, the controller 910 receives neural signal data from the subject via the input 916. These neural signal data are provided to the processor 912 where they are processed. For example, the processor 912 analyzes the neural signal data using, in part, a Bayesian optimization framework for determining and adjusting stimulation settings for the neurostimulation device.

Referring now to FIG. 10, an example neurostimulation device 1010 is shown. In the illustrated example, the neurostimulation device 1010 is configured as a deep brain stimulation (DBS) system capable of simultaneously recording neural activity and delivering neurostimulation to a target brain region to prevent anticipated pathological neural activity (e.g., epileptic seizure). The neurostimulation device 1010 includes one or more stimulating electrodes 1012 for stimulating populations of neurons in the target brain region and one or more recording electrodes 1014 for measuring neuronal activity in response to the delivered neurostimulation. The electrodes 1012, 1014 are typically implanted in the subject and connected via an insulated lead 1016 to a neurostimulator 1018. In some implementations, the lead 1016 runs under the skin of the head, neck, and shoulder and the neurostimulator 1018 is implanted to sit inferior to the clavicle. The neurostimulator 1018 includes a pulse generator 1020, a controller 1022 (e.g., controller 910), and a battery pack 1024 for powering the neurostimulation device 1010. The neurostimulator 1018 can also include a memory (e.g., memory 914) to store measured neural activity data and models for implementation on the controller 1022.

In operation, the neurostimulation device 1010 measures neuronal activity data by acquiring neural activity signals from the brain with the recording electrode(s) 1014. These neural activity signals (e.g., LFPs) are carried via lead 1016 to the neurostimulator 1018 where they are processed by the controller 1022 and/or transmitted to an external device for processing. The controller 1022 and/or external device analyzes the neural activity signals and determines a subject-specific neurostimulation setting using the methods described in the present disclosure. The selected stimulation signal is then generated by the pulse generator 1020 according to the subject-specific neurostimulation setting and delivered via the lead 1016 to the stimulating electrode(s) 1012, which delivers the neurostimulation to the target area.

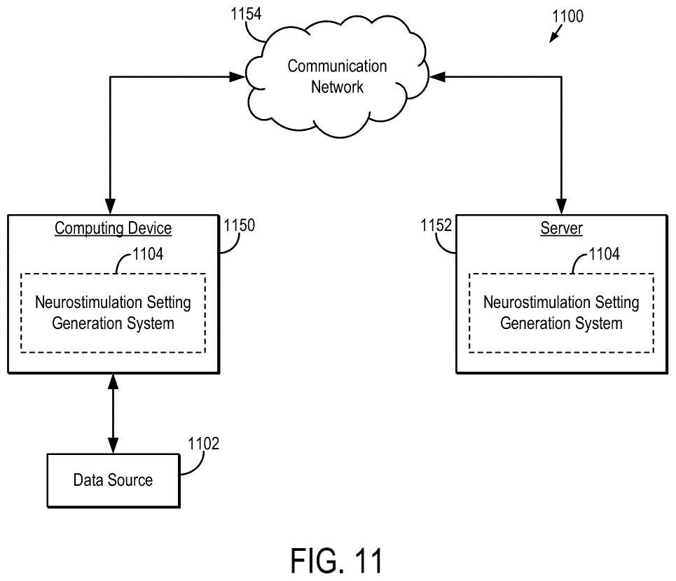

FIG. 11 shows an example of a system 1100 for selecting subject-specific neurostimulation settings in accordance with some embodiments described in the present disclosure. As shown in FIG. 11, a computing device 1150 can receive one or more types of data (e.g., neural activity) from data source 1102. In some embodiments, computing device 1150 can execute at least a portion of a neurostimulation setting generation system 1104 to select a subject-specific neurostimulation setting (e.g., that may optimize neurostimulation for the subject) from data received from the data source 1102.

Additionally or alternatively, in some embodiments, the computing device 1150 can communicate information about data received from the data source 1102 to a server 1152 over a communication network 1154, which can execute at least a portion of the neurostimulation setting generation system 1104. In such embodiments, the server 1152 can return information to the computing device 1150 (and/or any other suitable computing device) indicative of an output of the neurostimulation setting generation system 1104.

In some embodiments, computing device 1150 and/or server 1152 can be any suitable computing device or combination of devices, such as a desktop computer, a laptop computer, a smartphone, a tablet computer, a wearable computer, a server computer, a virtual machine being executed by a physical computing device, and so on. Additionally or alternatively, the computing device 1102 can be a controller for a medical device, such as a neuromodulation device. For example, the computing device 1102 can implement the controller 910 shown in FIG. 9.

In some embodiments, data source 1102 can be any suitable source of data (e.g., measurement data, processed measurement data), such as recording electrodes, another computing device (e.g., a server storing measurement data, images reconstructed from measurement data, processed image data), and so on. In some embodiments, data source 1102 can be local to computing device 1150. For example, data source 1102 can be incorporated with computing device 1150 (e.g., computing device 1150 can be configured as part of a device for measuring, recording, estimating, acquiring, or otherwise collecting or storing data). As another example, data source 1102 can be connected to computing device 1150 by a cable, a direct wireless link, and so on. Additionally or alternatively, in some embodiments, data source 1102 can be located locally and/or remotely from computing device 1150, and can communicate data to computing device 1150 (and/or server 1152) via a communication network (e.g., communication network 1154).

In some embodiments, communication network 1154 can be any suitable communication network or combination of communication networks. For example, communication network 1154 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 1154 can be a local area network, a wide area network, a public network (e.g., the Internet), a private or semi-private network (e.g., a corporate or university intranet), any other suitable type of network, or any suitable combination of networks. Communications links shown in FIG. 11 can each be any suitable communications link or combination of communications links, such as wired links, fiber optic links, Wi-Fi links, Bluetooth links, cellular links, and so on.

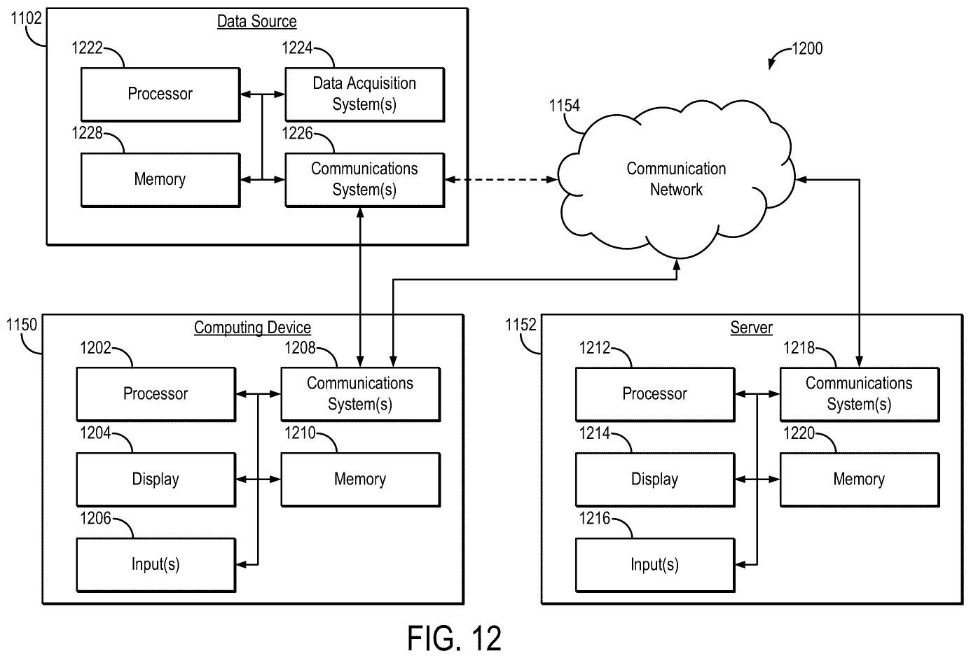

Referring now to FIG. 12, an example of hardware 1200 that can be used to implement data source 1102, computing device 1150, and server 1152 in accordance with some embodiments of the systems and methods described in the present disclosure is shown.

As shown in FIG. 12, in some embodiments, computing device 1150 can include a processor 1202, a display 1204, one or more inputs 1206, one or more communication systems 1208, and/or memory 1210. In some embodiments, processor 1202 can be any suitable hardware processor or combination of processors, such as a central processing unit (CPU), a graphics processing unit (GPU), and so on. In some embodiments, display 1204 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 1206 can include any suitable input devices and/or sensors that can be used to receive user input, such as a keyboard, a mouse, a touchscreen, a microphone, and so on.

In some embodiments, communications systems 1208 can include any suitable hardware, firmware, and/or software for communicating information over communication network 1154 and/or any other suitable communication networks. For example, communications systems 1208 can include one or more transceivers, one or more communication chips and/or chip sets, and so on. In a more particular example, communications systems 1208 can include hardware, firmware, and/or software that can be used to establish a Wi-Fi connection, a Bluetooth connection, a cellular connection, an Ethernet connection, and so on.

In some embodiments, memory 1210 can include any suitable storage device or devices that can be used to store instructions, values, data, or the like, that can be used, for example, by processor 1202 to present content using display 1204, to communicate with server 1152 via communications system(s) 1208, and so on. Memory 1210 can include any suitable volatile memory, non-volatile memory, storage, or any suitable combination thereof. For example, memory 1210 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 1210 can have encoded thereon, or otherwise stored therein, a computer program for controlling operation of computing device 1150. In such embodiments, processor 1202 can execute at least a portion of the computer program to present content (e.g., images, user interfaces, graphics, tables), receive content from server 1152, transmit information to server 1152, and so on. For example, the processor 1202 and the memory 1210 can be configured to perform the methods described herein (e.g., the method of FIG. 8).

In some embodiments, server 1152 can include a processor 1212, a display 1214, one or more inputs 1216, one or more communications systems 1218, and/or memory 1220. In some embodiments, processor 1212 can be any suitable hardware processor or combination of processors, such as a CPU, a GPU, and so on. In some embodiments, display 1214 can include any suitable display devices, such as an LCD screen, LED display, OLED display, electrophoretic display, a computer monitor, a touchscreen, a television, and so on. In some embodiments, inputs 1216 can include any suitable input devices and/or sensors that can be used to receive user input, such as a keyboard, a mouse, a touchscreen, a microphone, and so on.

In some embodiments, communications systems 1218 can include any suitable hardware, firmware, and/or software for communicating information over communication network 1154 and/or any other suitable communication networks. For example, communications systems 1218 can include one or more transceivers, one or more communication chips and/or chip sets, and so on. In a more particular example, communications systems 1218 can include hardware, firmware, and/or software that can be used to establish a Wi-Fi connection, a Bluetooth connection, a cellular connection, an Ethernet connection, and so on.

In some embodiments, memory 1220 can include any suitable storage device or devices that can be used to store instructions, values, data, or the like, that can be used, for example, by processor 1212 to present content using display 1214, to communicate with one or more computing devices 1150, and so on. Memory 1220 can include any suitable volatile memory, non-volatile memory, storage, or any suitable combination thereof. For example, memory 1220 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1220 can have encoded thereon a server program for controlling operation of server 1152. In such embodiments, processor 1212 can execute at least a portion of the server program to transmit information and/or content (e.g., data, images, a user interface) to one or more computing devices 1150, receive information and/or content from one or more computing devices 1150, receive instructions from one or more devices (e.g., a personal computer, a laptop computer, a tablet computer, a smartphone), and so on.

In some embodiments, the server 1152 is configured to perform the methods described in the present disclosure. For example, the processor 1212 and memory 1220 can be configured to perform the methods described herein (e.g., the method of FIG. 8).

In some embodiments, data source 1102 can include a processor 1222, one or more data acquisition systems 1224, one or more communications systems 1226, and/or memory 1228. In some embodiments, processor 1222 can be any suitable hardware processor or combination of processors, such as a CPU, a GPU, and so on. In some embodiments, the one or more data acquisition systems 1224 are generally configured to acquire data and can include one or more recording electrodes. Additionally or alternatively, in some embodiments, the one or more data acquisition systems 1224 can include any suitable hardware, firmware, and/or software for coupling to and/or controlling operations of the recording electrode(s) and/or a controller that controls operation of the recording electrode(s). In some embodiments, one or more portions of the data acquisition system(s) 1224 can be removable and/or replaceable.

Note that, although not shown, data source 1102 can include any suitable inputs and/or outputs. For example, data source 1102 can include input devices and/or sensors that can be used to receive user input, such as a keyboard, a mouse, a touchscreen, a microphone, a trackpad, a trackball, and so on. As another example, data source 1102 can include any suitable display devices, such as an LCD screen, an LED display, an OLED display, an electrophoretic display, a computer monitor, a touchscreen, a television, etc., one or more speakers, and so on.

In some embodiments, communications systems 1226 can include any suitable hardware, firmware, and/or software for communicating information to computing device 1150 (and, in some embodiments, over communication network 1154 and/or any other suitable communication networks). For example, communications systems 1226 can include one or more transceivers, one or more communication chips and/or chip sets, and so on. In a more particular example, communications systems 1226 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1228 can include any suitable storage device or devices that can be used to store instructions, values, data, or the like, that can be used, for example, by processor 1222 to control the one or more data acquisition systems 1224, and/or receive data from the one or more data acquisition systems 1224; to generate images from data; present content (e.g., data, images, a user interface) using a display; communicate with one or more computing devices 1150; and so on. Memory 1228 can include any suitable volatile memory, non-volatile memory, storage, or any suitable combination thereof. For example, memory 1228 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1228 can have encoded thereon, or otherwise stored therein, a program for controlling operation of data source 1102. In such embodiments, processor 1222 can execute at least a portion of the program to generate images, transmit information and/or content (e.g., data, images, a user interface) to one or more computing devices 1150, receive information and/or content from one or more computing devices 1150, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for determining neurostimulation settings for a neurostimulation device, the method comprising:
(a) measuring neural activity in a subject using a recording electrode while delivering neurostimulation using a stimulating electrode, wherein the neurostimulation is delivered according to a plurality of different neurostimulation settings, wherein each of the plurality of different neurostimulation settings comprises a different set of stimulation parameters, wherein each different set of stimulation parameters comprises different values for a stimulation frequency, a pulse width, and an amplitude;
(b) determining, using a computing device, a subject-specific neurostimulation setting based on the measured neural activity by:
estimating a power for each neurostimulation setting from the neural activity measured while neurostimulation was being delivered with that neurostimulation setting;
generating a response surface from the power estimated for each neurostimulation settings, wherein the response surface indicates estimated power as a function of the neurostimulation settings;
selecting the subject-specific neurostimulation setting based on the response surface; and
(c) outputting the subject-specific neurostimulation setting from the computing device to the neurostimulation device.

2. The method of claim 1, wherein the amplitude in each of the different sets of stimulation parameters is selected to maintain a constant energy delivery across the plurality of different neurostimulation settings based on the different values for the stimulation frequency and the pulse width in each of the different sets of stimulation parameters.

3. The method of claim 2, wherein the different values for the stimulation frequency and the pulse width are systematically adjusted between the different sets of stimulation parameters according to a search grid.

4. The method of claim 1, wherein the power is estimated for each neurostimulation setting from neural activity measured in a frequency band.

5. The method of claim 4, wherein the frequency band is 1-80 Hz.

6. The method of claim 1, wherein the subject-specific neurostimulation setting is selected based on a Bayesian optimization using the response surface.

7. The method of claim 6, wherein the subject-specific neurostimulation setting is selected to minimize neural activity in a particular region of the subject's brain.

8. The method of claim 7, wherein the particular region of the subject's brain comprises a thalamus.

9. The method of claim 1, wherein the neural activity comprises thalamic activity recorded by the recording electrode from a thalamus of the subject.

10. The method of claim 1, wherein the neural activity comprises changes in neural activity in response to the neurostimulation being delivered with the different neurostimulation settings.

11. A non-transitory computer-readable storage medium having stored thereon instructions that when executed by a processor cause the processor to:
(i) repeat control a neurostimulation device to deliver neurostimulation to a subject using a stimulating electrode, wherein the neurostimulation is delivered according to neurostimulation settings comprising a stimulation frequency, a pulse width, and an amplitude;
(ii) control the neurostimulation device to measure neural activity data from the subject using a recording electrode, wherein the neural activity data comprise local field potentials measured in response to the delivered neurostimulation;
(iii) repeat steps (i) and (ii) while adjusting the neurostimulation settings by adjusting at least one of the stimulation frequency, the pulse width, or the amplitude;
(iv) generate a response surface from the neural activity data, wherein the response surface indicates measured neural activity as a function of the neurostimulation settings;
(v) determine an updated neurostimulation setting based on a Bayesian optimization using the response surface; and
(vi) store the updated stimulation setting in a memory of the neurostimulation device.

12. The non-transitory computer-readable storage medium of claim 11, wherein adjusting the neurostimulation settings comprises adjusting at least one of the stimulation frequency or the pulse width and selecting the amplitude to maintain a constant energy delivery across the neurostimulation settings based on adjusted stimulation frequency or pulse width.

13. The non-transitory computer-readable storage medium of claim 11, wherein adjusting the neurostimulation settings comprises systematically adjusting the stimulation frequency and pulse width according to a search grid.

14. The non-transitory computer-readable storage medium of claim 11, wherein generating the response surface comprises estimating a power from the neural activity data and generating the response surface based on the estimated power and the neurostimulation settings.

15. The non-transitory computer-readable storage medium of claim 14, wherein the power is estimated from neural activity data measured in a frequency band.

16. The non-transitory computer-readable storage medium of claim 15, wherein the frequency band is 1-80 Hz.

17. The non-transitory computer-readable storage medium of claim 11, wherein the updated neurostimulation setting is determined to minimize neural activity in a brain region.

18. The non-transitory computer-readable storage medium of claim 17, wherein the brain region is a thalamus.
